(19) United States
(12) Patent Application Publication
(10) Pub. No.: US 2012/0316077 A1
(43) Pub. Date: Dec. 13, 2012

(54) System And Method For Detection And Analysis Of A Molecule In A Sample

(76) Inventors: Charles Greef, Louisville, CO (US); Michael J. Lochhead, Boulder, CO (US)

(21) Appl. No.: 13/491,380

(22) Filed: Jun. 7, 2012

Related U.S. Application Data
(60) Provisional application No. 61/494,347, filed on Jun. 7, 2011.
(60) Provisional application No. 61/609,851, filed on Mar. 12, 2012.

(51) Int. Cl. G01N 21/64; C40B 30/04; G01N 33/53

(52) U.S. Cl. 506/9; 436/501

(57) ABSTRACT

Disclosed here is a system and method for analyzing and/or detecting one or more target analytes in a sample by using a competitive assay. A standard curve may be constructed using known amounts of a molecule that is identical or substantially identical to the target analyte. Signals obtained from the target analyte can be compared against the standard curve in order to determine the level of the target analyte in the sample. The disclosed methods may be used in a multiplexed analyte detection and quantitation system.

### RELATED APPLICATIONS

This application claims priority to U.S. Provisional Patent Application No. 61/494,347, filed Jun. 7, 2011 and to U.S. Provisional Patent Application No. 61/609,851, filed Mar. 12, 2012. All of the aforementioned applications are incorporated by reference into the present application in their entireties and for all purposes.

### BACKGROUND

I. Field of the Invention

The present disclosure pertains to identification and detection of an analyte or an object in a sample. More particularly, the disclosure relates to a system and methods for detecting and/or measuring small molecules, proteins or other molecules in a sample.

II. Description of Related Art

Competitive assays have been widely used in the medical and scientific fields for detection of molecules. In a competitive immunoassay, for example, a target analyte (e.g., an antigen) “competes” with the binding of a specific antibody to an immobilized antigen, typically in a microtiter plate well. In indirect competitive immunoassays, specific antibodies are mixed with a sample and the mixture is then allowed to interact with antigens immobilized on a solid surface. Wash steps are then performed in order to remove loosely bound antibodies and excess sample. Subsequently, a secondary antibody (also called a detection antibody) is added. The secondary antibody is typically conjugated with an excitable tag such as fluorophore, or an enzyme that will be used to generate detectable signal. A common format is to use an anti-species antibody (e.g., anti-mouse Ab) conjugated to horseradish peroxidase as the secondary antibody conjugate. In a subsequent step, enzyme substrate is added to generate detectable signal. In a competitive immunoassay, the absence of target analyte results in presence of detectable signal (see FIG. 1). When target analyte is present, it binds to the specific antibody and competes with binding to surface antigen, resulting in decreased signal or absence of signal relative to the sample with no target analyte.

Conventional competitive immunoassays have certain limitations. For instance, conventional assays typically require a skilled operator and auxiliary equipment (pipetters, plate washers, etc.) to perform the multiple assay steps. Assay protocols frequently have multiple timed steps. The detection of multiple different target analytes typically requires multiple parallel assays, which increase the amount of sample, reagent, and processing required to complete an assay. Further, conventional assays typically require refrigeration or biological components and therefore restrict use to laboratory settings. Conventional assays typically require too many steps and too much user interaction in order to complete a competitive assay. Conventional assays also require substantial amount of sample for each assay. Most traditional assays do not have the capability to simultaneously detect multiple target analytes in a single, small sample, with minimal user interaction.

### SUMMARY

The present instrumentalities advance the art by providing an improved assay system that solves some of the problems in the field. In one embodiment, the system may include a device and a reader instrument. Examples of the device may include but are not limited to a cartridge, a substrate, a channel, or other solid supports capable of transmitting light and holding the sample. In another embodiment, the reader instrument may be capable of detecting and measuring light signals emitted from the device.

In another embodiment, the device may contain a waveguide. In another embodiment, the device may contain a first substrate and a second substrate. In another embodiment, the first substrate or the second substrate may contain a planar waveguide. In another embodiment, the planar waveguide may be a multi-mode planar waveguide. In another embodiment, the planar waveguide may have an integrally-formed lens. In another embodiment, the planar waveguide may contain at least a first outer surface and a first inner surface, while the second substrate may contain at least a second outer surface and a second inner surface. In another embodiment, the first inner surface and the second inner surface are spaced apart from each other, wherein the first inner surface and the second inner surface at least partly define a sample chamber that may hold or confine the entire sample or a portion thereof. In another embodiment, the first substrate and the second substrate are positioned such that at least a section of the first inner surface and a section of the second inner surface are apart from each other at a distance wherein this section of the first inner surface and this section of the second inner surface at least partly define a sample chamber for holding or confining the sample or at least a portion thereof. In another aspect, the device may have an inlet port and an outlet port, and the inlet and outlet ports may be both connected with the sample chamber.

In another embodiment, the present disclosure provides a system and method for analyzing and/or detecting one or more target analytes (or molecules) in a sample by using a competitive assay. The sample may contain one type of target analytes, or it may contain different types of target analytes. The target analytes may be a protein, an antibody, an antigen, an aptamer, a polysaccharide, a sugar molecule, a carbohydrate, a lipid, an oligonucleotide, a polynucleotide, a synthetic molecule, a small molecule, an inorganic molecule, an organic molecule, or combination thereof.

In another embodiment, a system and method are disclosed for determining the amount of one or more target analytes in a sample. In step (a), the sample or a portion thereof is incubated with one or more types of labeling molecules for a period of time to form a mixture. The one or more types of labeling molecules may be selected such that at least one of the labeling molecules specifically binds at least one of the one or more target analytes. Thus, if the sample contains one or more target analytes, at least one of the labeling molecules would bind to at least one of the target analytes in the sample. If the sample does not contain the one or more target analytes that specifically binds at least one of the labeling molecules, the labeling molecules would not specifically bind to any target analytes in the sample. In step (b), the mixture of step (a) may be caused to be in contact with one or more types of capture molecules immobilized on a surface of the device, wherein the one or more types of capture molecules bind to the one or more types of labeling molecules. In step (c), the intensity of a first signal emitted from the surface of the device may be measured to determine the amount of the one or more target analytes in the sample.

In one aspect, step (a) and step (b) of the process may occur in the same device. In another aspect, step (a) occurs before step (b) and no intervention or action by an operator is required between step (a) and step (b). In another aspect, steps (a)-(c) may be performed without conducting a wash step during any steps or between any steps. In one embodiment, steps (a) and (b) may be merged such that the labeling molecules may contact the target analytes and the capture molecules at the same time.

For purpose of this disclosure, the term “immobilized” may be used to refer to the condition of being attached to another object or surface with a substantial affinity via chemical or physical interaction. A molecule may be immobilized to a surface or an object directly by binding to the surface or object directly or it may be immobilized to the surface or object indirectly through interaction (e.g., bonding, conjugation, or binding, among others) with another molecule.

The labeling molecule may be any molecules that specifically binds a target molecule either in solution or on a surface, having a specific binding interaction with the target analyte(s). A labeling molecule is also referred to as a detecting reagent throughout this disclosure. Examples of labeling molecules may include but are not limited to antibodies, aptamers, affibodies, proteins, small organic molecules, carbohydrate molecules, lipid molecules, polynucleotides, other molecules capable of binding one or more target analytes, or combination thereof. In another embodiment, the labeling molecule may non-specifically interact or bind with the target analyte(s). In one embodiment, the labeling molecule is an antibody. In another embodiment, the labeling molecule is an antibody conjugated with an excitable tag, such as a fluorophore.

The device may have a plurality of capture molecules pre-attached to its surface, for example, on the first inner surface of the device. The plurality of capture molecules may be of different types, with each type binding to one or more types of labeling molecules. The capture molecules may be the same as the target analyte(s) or may be different from the target analyte(s). Examples of capture molecules may be a protein, an antibody, an antigen, an aptamer, a polysaccharide, a sugar molecule, a carbohydrate, a lipid, an oligonucleotide, a polynucleotide, a synthetic molecule, a small molecule, an inorganic molecule, an organic molecule, or combination thereof. In one embodiment, the capture molecule is an antigen.

In one embodiment, two or more different types of labeling molecules may be used. The two or more different types of labeling molecules may have specific affinity to two or more different types of target analytes, respectively. Two or more different types of capture molecules may be immobilized in a spatial array of spots on a surface of the device. The spatial array may contain at least two spots, four spots, eight spots, or sixteen spots, or even more. Except for those spots used as negative or positive controls, each spot may contain a capture molecule of one specific type which binds specifically to a specific type of labeling molecule. Because each specific type of labeling molecule may bind to a specific target analyte in the sample, the spatial array of spots may be used to analyze multiple target analytes simultaneously.

In another embodiment, the labeling molecules may contain at least two different antibodies with substantial but different affinities to the same target analyte. The results obtained on the same target analyte using these different antibodies may be compared.

The disclosed method may further include a step (d) of comparing the intensity of the first signal obtained in step (c) against the intensity of a second signal obtained from a calibrating molecule having known amount. In one aspect, the intensity of the first signal may be compared against the intensity of a plurality of second signals obtained from the same calibrating molecules having a series of known amounts. These plurality of second signals may be plotted into a standard curve.

In one aspect, the calibrating molecule is the same molecule as the target analyte. In another aspect, the calibrating molecules and the target analytes may have substantially identical binding affinity with the labeling molecules. In another aspect, the capture molecules and the target analytes may have substantially identical binding affinity with the labeling molecules. The capture molecules and the target analytes may bind to the same binding site(s) on the labeling molecules such that the capture molecules and the target analytes compete against each to bind to such binding site(s).

In performing step (c) of the disclosed method, the signal from each spot of the spatial array may be measured independently, and signal intensity of each spot may be compared against different standard curves, wherein each standard curve is specific to each target analyte.

The sample may contain at least one object to be analyzed (also referred to as a “target analyte”). In a competitive assay, the target analyte may bind to the labeling molecules, thereby reducing the number of labeling molecules available for binding with other binding partners. The mixture containing the labeling molecules and the sample may be loaded onto a device, such as a cartridge. In one aspect, the labeling molecules may be conjugated with one or more excitable tags (e.g., a fluorophore), such that no secondary labeling is necessary. In another aspect, the labeling molecules may be conjugated to functional tags such as magnetic particles or particles that sediment in a gravitational field. In another aspect, the labeling molecules may be labeled with a second labeling molecule such as a secondary antibody. The second labeling molecule may be conjugated with one or more excitable tags.

In another embodiment, the method may also include a step of allowing the mixture to be in contact with the first inner surface. In one aspect, the labeling molecules may accumulate or sediment at the first inner surface through the force of gravity. In another aspect, the labeling molecules may bind to the capture molecules that are pre-attached to the surface. In one embodiment, only labeling molecules that are not bound to a target analyte is immobilized and attached to a capture molecule at the first inner surface.

In one embodiment, the labeling molecules may contain an excitable tag. In another embodiment, the labeling molecules may be further labeled with a second labeling molecule having an excitable tag. The disclosed method may also include a step of illuminating the labeling molecules immobilized at the first substrate using one or more light conditions to cause the tagged labeling molecules to emit fluorescent light. The fluorescent light may be captured and analyzed to determine the amounts of the labeling molecules at the first substrate. In another aspect, the method may include a step of providing light from a light source to illuminate the refractive volume of the device, wherein the light is coupled to the planar waveguide via the refractive volume.

In one embodiment, the target analyte and the capture molecule may compete against each other for binding to the labeling molecule. When the amount of a target analytes is higher in the sample, more labeling molecules are bound to these target analytes, and fewer labeling molecules are available to bind to the capture molecules. Thus, the intensity of the signal obtained from the labeling molecules that are bound to the capture molecules may be inversely proportional to the concentration of the target analytes in the sample. In one aspect, the presence of a specific target analyte in a sample may inhibit binding of at least one labeling molecule to at least one capture molecule, such that the presence of the target analyte results in a decreased first signal intensity as measured in step (c) relative to the intensity obtained when a sample containing no target analyte is used. In another aspect, such decrease in first signal intensity due to the presence of a specific target analyte may be quantitatively related to the amount of the specific target analyte in the sample.

In one embodiment, the labeling molecule may be a polyclonal or monoclonal antibody and the target analyte and the capture molecule may be an antigen. In another embodiment, the capture molecule and the target analyte may be the same antigen. In another embodiment, the capture molecule and the target analyte may be different antigens but the capture molecules and the target analytes may bind to the same binding site on the labeling molecule. In one aspect, when the labeling molecule is an antibody, the capture molecules and the target analytes may bind to the same binding site on the labeling molecule. In another aspect, the labeling molecules, capture molecules and the target analytes may be selected such that all binding sites on the labeling molecule that bind to the capture molecules also bind to the target analytes.

In one embodiment, the intensity of a first signal emitted by the labeling molecules that are bound to the capture molecules may be measured. In one aspect, the signal emitted by the labeling molecules may be a light signal, such as a fluorescent light signal. In another aspect, a wash buffer may be applied to the cartridge to wash off unbound labeling molecules prior to measuring the intensity of the first signal.

In one aspect, the signal intensity obtained from the labeling molecules (also referred to as “first signal”) as described above may be compared against signal intensity obtained from a calibrating molecule having a known amount (also referred to as “second signal”). In another aspect, the intensity of the first signal may be compared against the intensity of a second signal obtained from a calibrating molecule having known amount. In another aspect, the intensity of more than one second signal may be obtained from a plurality of calibrating molecules having known amounts, and the intensity of the first signal may be compared against the intensity of these second signals. The intensity of the second signals obtained from the plurality of calibrating molecules may be plotted against the known amounts of the calibrating molecules to construct a standard curve. In another embodiment, the intensity of the first signal may be compared against the standard curve in order to determine the amount (or concentrations) of the target analytes in the sample.

In one embodiment, the calibrating molecules may be molecules that bind to the labeling molecule with substantially identical binding affinity as the target analytes. In another embodiment, the calibrating molecules may be the same molecules as the target analytes except that the amounts of the calibrating molecules are known or, in other words, pre-determined.

In one embodiment, the binding between the labeling molecules and the calibrating molecules may take place under condition that is similar to the condition under which the sample is incubated with the labeling molecules. In another embodiment, the binding between the labeling molecules and the calibrating molecules may take place under the same condition as the condition under which the sample is incubated with the labeling molecules. For instance, if a blood sample is to be assayed, the calibrating molecules may be added into the same or similar blood sample that is known not to contain any target analytes. Thus, when the calibrating molecules are incubated with the labeling molecules, the incubation conditions are similar or identical to the conditions when the target analytes are incubated with the labeling molecules.

In one embodiment, the plurality of calibrating molecules may be immobilized on the same surface as the capture molecules, and the intensity of a first signal obtained from a target analyte may be compared directly with the series of second signals obtained from the plurality of calibrating molecules, providing in-assay calibration. For example, an array with capture molecules to antibodies A (test antibody) and B (calibrator) can be compared in the absence of target molecule A, such that a ratio of signal between antibodies A and B in the absence of target molecule A is determined. Then, when a sample containing target molecule A is tested, the signal from antibody A, which will be lower in proportion to the concentration of target molecule A, is compared to the signal from antibody B in a ratiometric measurement so that the ratio of signal from antibody A to antibody B in the presence of target molecule A is indicative of the concentration of target molecule A.

In one aspect, capture molecules to antibodies A and B are printed as separate spots in the spatial array, and use a common fluorescent label on antibodies A and B, and are measured simultaneously. In another aspect, capture molecules to antibodies A and B are printed as mixtures to the same spot, and antibodies A and B are labeled with fluorescent labels of different spectral excitement and emission ranges, so that a sequential image acquisition method wherein signal from antibody A from channel 1 is collected, then signal from antibody B from channel 2 is collected, and ratiometric analyses provide quantitative information about the concentration of target molecule A.

In another embodiment, the device may contain multiple, parallel assay channels that may be used to generate calibration curves. In one embodiment, the device is a three channel device, with one channel dedicated to a positive control sample, a second channel dedicated to a negative control sample, and the third channel dedicated to the unknown sample. Additional channels could be added to provide more quantitative calibration data. In one embodiment, all channels are analyzed simultaneously in a reader instrument.

In another embodiment, more than one target analyte in a sample may be assayed simultaneously. Different labeling molecules may be mixed with the same sample wherein the different target analytes bind to their respective binding partners, or in this case, the labeling molecules. Specific capture molecules may be pre-printed on the first inner surface of the cartridge in a two dimensional spatial array, wherein each specific capture molecule can bind to different labeling molecules specifically. The terms “spot,” “capture spot,” and “array feature” are used to describe the elements of the spatial array.

In another embodiment, the different labeling molecules do not cross-react with the different target analytes and do not interfere with the association between other labeling molecules and their respective binding partners.

The competitive assay using the system disclosed here is capable of detecting extremely low concentration of target analytes within a wide dynamic range. The dynamic range of an assay is defined as the range of target analytes levels or concentration within which quantification is accurate. In one embodiment, the dynamic range of the disclosed assay is in the range of about 0.001 ng/ml to about 1,000,000 ng/ml, where the concentration expressed in “ng/ml” is the concentration of the target analyte in a biological sample. In another embodiment, the dynamic range of the disclosed assay is in the range of about 0.01 ng/ml to about 1,000 ng/ml. In another embodiment, the dynamic range of the disclosed assay is in the range of about 0.1 ng/ml to about 100 ng/ml. In another embodiment, the dynamic range of the disclosed assay is in the range of about 0.2 ng/ml to about 10 ng/ml. In another embodiment, the dynamic range of the disclosed assay is in the range of about 10 ng/mL to 1,000,000 ng/mL. In another embodiment, the dynamic range of the disclosed assay is in the range of about 0.001 ng/mL to 1 ng/mL. The dynamic range of the disclosed assay may depend upon many factors, such as, for example, the amount of labeling molecules, the amount of capture molecules on the cartridge, the conditions of the incubation buffer (e.g., salt and pH), the binding affinity between the labeling molecules and the target analytes relative to the binding affinity between the labeling molecules and the capture molecules. All these factors may be adjusted to shift the dynamic range according to specific needs. Under certain circumstances, two or more factors listed above may be adjusted simultaneously in order to achieve the desired dynamic range. For instance, the concentration of the labeling molecules and/or the amount of the capture molecules may be adjusted to shift the dynamic range of the disclosed assay.

In another embodiment, when the concentration of a target analyte is predicted or determined to be outside the dynamic range of an assay, the sample may be diluted or concentrated before an assay. For instance, when the concentration of a target analyte is high, the sample may be diluted before incubation with the labeling molecules. At the end of the assay, the actual analyte concentration may be calculated from the measured concentration based on the factor of dilution.

By way of example, when the labeling molecule is an antibody, it may be diluted by from 1×10⁶ to 1×10³ folds before being incubated with the sample. In another aspect, antibody may be diluted by from 1×10⁴ to 1×10³ folds before being incubated with the sample. In another aspect, the antibody may be diluted by about 1×10⁴ or about 1×10³ fold before being incubated with the sample. Typically, in order to raise the upper limit of the dynamic range, more labeling molecules and/or more capture molecules may be used.

Alternate surface chemistries may be used to enable higher sensitivity and/or wider dynamic range. In one aspect, variations to surface chemistry treatments may enable alternate surface binding mechanisms which, in turn, may result in higher surface loading, higher signal, less variable signal, lower background, improved flow dynamics. These treatments may include but are not limited to: epoxy silanes, aldehyde silanes, carboxylate silanes, Ni-NTA, NHS ester, S-NHS ester, CDI, methacrylate, PEG modified with any specific attachment chemistry, streptavidin, protein A.

In another embodiment, the dynamic range may be extended through titrated print concentrations (i.e. surface densities) of surface bound capture molecules in capture spots. For example, the dynamic range of target analyte detection and/or quantitation may be extended by printing different concentrations of capture molecules in different capture spots in the spatial array, so that the measured signal from bound labeling molecules is different on differentially printed capture spots, and differentially printed capture spots may react as a function of the concentration of target in the sample.

In another embodiment, variations to the assay procedure may also allow for different assay functions according to the desired purpose. For instance, one procedure may be performed for a high sensitivity application, another procedure may be performed for a quantitative application in a certain range, and yet another procedure may be performed for widest quantitative dynamic range.

In another embodiment, a mixture of antibodies with different affinities to the target may be used to extend the dynamic range. Lower affinity antibodies may react and be quantitative at higher target concentration, while higher affinity antibodies would work at the lowest limit of detection.

In another embodiment, the rate of signal accumulation on a capture spot may be measured and compared to a standard or a standard curve or a normalized responder on the cartridge, such that the rate of signal accumulation may be quantitatively correlated to target concentration in the sample.

In another embodiment, competition assays of target binding antibodies matching antigen conjugates may be bound to the surface, in which the antibody is fluorescently labeled. When mixed with a sample of unknown target concentration and applied to the cartridge, the fluor-labeled antibody may bind to antigen conjugate content on the surface in inverse proportion to the concentration of target in the sample. Because the antibody is labeled, no further reagent additions are required.

In one embodiment, the labeling molecule may be an antibody. In another embodiment, competition assays may be carried out using labeling molecules other than antibodies, such as aptamers, affibodies, or another molecule that specifically binds a target molecule either in solution or on a surface, such that it can be used in a competitive assay format.

In one embodiment, in-array or in-cartridge normalization or referencing spots may be used to eliminate the need to run a standard curve. It may be useful to have an in-array method to establish the baseline signal from which to compare sample response, thereby eliminating the need to build a standard curve of known zero target concentration. A second or orthogonal antibody/antigen conjugate system may be used on the cartridge wherein the response recorded from the second antibody antigen conjugate system is unaffected by presence of target, and matched and quantitatively correlated to the first antibody. Differences of response in the presence of sample are indicative of presence of target molecule to the first antibody and quantitatively correlated to the concentration of target molecule in the sample.

In another embodiment, control features may be incorporated in the cartridges to allow for quantitative comparison of responses from different cartridges, such that variations in response resulting from systematic or manufacturing variability are scaled to a common factor for equalized measurement. Methods of processing data responses may be implemented to scale signals to a common reference between cartridges allowing quantitative response comparison.

In another embodiment, various methods may be used to increase throughput and/or automate operation of assays. Lyophilized reagent mixtures that are pre-measured into reaction tubes may be used, allowing addition of a fixed volume of water and/or sample prior to application to the cartridge. Lyophilized reagent mixtures that are built into localized areas of the cartridge may be used, such that addition of a sample containing unknown concentrations of target re-hydrates the reagent mixture prior to the mixture flowing over the analytical area, so that only one action of material introduction to the cartridge is required to perform the assay. Onboard liquid reservoirs (e.g., blister packs) that release liquid into fluidic channel upon user action may also be employed. In one aspect, a cartridge having a sample inlet may be used and the labeling molecules may be incorporated in the cartridge. By way of example, the dried labeling molecules may be placed near the inlet such that a sample containing unknown concentration of target analytes may contact the labeling molecules after the sample is loaded onto the cartridge, but prior to the sample contacting the immobilized capture molecules. The sample may rehydrate the dried labeling molecules and form a mixture containing both the labeling molecules and the target analytes. The mixture may then make contact with the capture molecules that are immobilized on the cartridge.

In another embodiment, the competitive assay may be carried out by printing fluorescently labeled antibody on the waveguide surface. Assay component A may be a protein carrier conjugated with 1) Target and 2) Quencher. The printed antibodies are specific to the target in component A and also specific to the target in the sample. In the absence of free target in a sample, assay component A binds the fluor-labeled antibody on the surface and quenches fluorescent signal, resulting in no fluorescent signal. In the presence of free target in the sample, free target competes with component A for binding to antibody on surface, and when it does bind to the immobilized antibodies, fluorescence is uninhibited or inhibited to a lesser extent. Thus the assay is competitive, with presence of target resulting in increase of signal proportionate to concentration of target. A standard curve may be constructed as described above.

In another embodiment, an assay may be performed in which Assay component A is a protein carrier conjugated with 1) Fluorescent dye and 2) Target, and component A is printed to the surface of the cartridge. Antibody which is chemically conjugated with Quencher is in sample mix. In the absence of target, Antibody-quencher binds component A on surface and quenches fluorescence. In the presence of target, target binds antibody-quencher which may not bind component A, allowing fluorescence. Thus the assay is competitive, with presence of target resulting in increase of signal proportionate to concentration of target.

### BRIEF DESCRIPTION OF THE DRAWINGS

FIG. 1 illustrates the steps of an indirect competitive fluorescence immunoassay.

FIG. 2 illustrates a direct competitive assay, in accordance with an embodiment.

FIG. 3 illustrates a cross-sectional view of an assay system including a waveguide with an integrated lens, illumination, and imaging system, in accordance with an embodiment.

FIG. 4 shows a flow chart illustrating one of the competitive assay processes, in accordance with an embodiment.

FIG. 5 is a graph showing the measured fluorescence signal from a competitive detection assay, in accordance with an embodiment.

It is noted that, for purposes of illustrative clarity, certain elements in the drawings may not be drawn to scale.

### DETAILED DESCRIPTION

The present disclosure provides a system and method for detecting and characterizing an analyte or an object in a sample. The disclosed system and method may also enable simultaneous detection of multiple analytes in one single step. In one embodiment, a sample or a portion thereof may be loaded onto a cartridge and results on multiple analytes may be obtained from the cartridge reader within a short period of time. In another embodiment, minimal or no user intervention or action is required between sample input and readout of results. In another embodiment, a sample may be added to a device in a single user interaction, and results for multiple target analytes may be delivered from the device in a rapid and inexpensive manner.

In one embodiment, the target analyte may be a protein, an antigen, an antibody, a food allergen, a hormone, an antibiotic or antibiotic residue, a toxin, a pesticide, a pollutant. In another embodiment, the target analyte may be a molecule in or on the surface of a pathogen. Such a molecule may be a protein, a polynucleotide, a lipid or sugar molecule. By way of example, the pathogen may be a bacterium, a virus, a fungus, among others. Presence of such a molecule may be indicative of the presence of a pathogen.

In one embodiment, a labeling molecule that generates a quantifiable signal may be used in a competitive assay. In another embodiment, the sample may be incubated with one or more labeling molecules to create a mixture. At least one of the labeling molecules may bind to the one or more target analytes. In one aspect, the labeling molecules may be a heterogeneous population of different molecules. In another aspect, the labeling molecules may be a homogeneous population containing the same molecules, such as, the same antibody molecules.

Incubation of the sample and the labeling molecules may be carried out for a short period of time, such as, for example, 1 second, 10 seconds, 30 seconds, 1 minute, 5 minutes, 20 minutes, or longer. In one embodiment, the binding between the target analytes and the labeling molecules may effectively be instantaneous.

In one embodiment, the labeling molecules may be pre-tagged with a detectable tag before the labeling molecules are incubated with the sample. Examples of detectable tags may be an excitable tag, such as a fluorescence tag. In another embodiment, a second labeling molecule, such as a tagged secondary antibody that binds specifically to the labeling molecules, may be added into the chamber to label the labeling molecules that are already bound to the capture molecules. Signals from the labeling molecules may be measured whose intensity is proportional to the number of labeling molecules that are bound to the surface of the chamber. In one embodiment, the labeling molecules may be pre-tagged or labeled with a fluorescence tag and fluorescent light intensity emitted by the labeling molecules may be measured by a light-detecting means, similar to the system described in U.S. Patent Application Publication 2010/0220318.

In exemplary embodiments, excitable tags may be used as detection reagents in assay protocols. Exemplary tags may include, but are not limited to, fluorescent organic dyes such as fluorescein, rhodamine, and commercial derivatives such as Alexa dyes (Life Technologies) and DyLight products; fluorescent proteins such as R-phycoerythrin and commercial analogs such as SureLight P3; luminescent lanthanide chelates; luminescent semiconductor nanoparticles (e.g., quantum dots); phosphorescent materials, and microparticles (e.g., latex beads) that incorporate these excitable tags. For the purpose of this disclosure, the term “fluorophore” is used generically to describe all of the excitable tags listed here. The terms “fluorophore-labeled,” “fluor-labeled,” “dye-labeled,” “dye-conjugated,” “tagged,” and “fluorescently tagged” may be used interchangeably in this disclosure.

The embodiments described herein may be applicable to assays beyond fluorescence-based signal transduction. For example, the methods and systems may also be compatible with luminescence, phosphorescence, and light scattering based signal transduction.

In one embodiment, two-color fluorescence microscopy based on planar waveguide illumination and differential immunostaining can be used in connection with the use of two or more different labeling molecules, such as two or more different primary antibodies.

Plural or singular forms of a noun may be used interchangeably unless otherwise specified in the disclosure.

In one embodiment, the sample may be a body fluid obtained from a subject. Examples of the samples suitable for the instant system may include but are not limited to whole blood sample, plasma, serum, sputum, bronchoalveolar lavage samples or aspirates, nasopharyngeal swabs, nasal swabs, cerebrospinal fluid (“CSF”), saliva, lymphatic fluid, amniotic fluid, ascites fluid, urine or a combination thereof. In another embodiment the sample may include but is not limited to cultured cells, cell preparations, cell extracts, culture media or combinations thereof. In another embodiment, the sample can be an environmental sample, waste water, industrial waste, food, agriculture products, meat product, or combination thereof. In the case where the target analyte is in a solid material, a liquid wash may be used to obtain a fluid sample from the solid material.

Another feature of the present disclosure is that devices (e.g., cartridges) are processed independently of the reader instrument, enabling batch mode processing of cartridges. This provides a significant throughput advantage over competing technologies in which the instrument is occupied during cartridge processing. In one embodiment, the assay time on the reader instrument is less than 4 minutes, enabling up to 15 samples to be processed per hour on the reader.

Another feature of the disclosed methods and systems is that a relatively small amount of the sample is required for each assay. In the context of blood based assays, it is desirable for the system to be compatible with both venous whole blood and capillary (finger stick) whole blood. In an embodiment, the sample has a specific volume in the range of 0.1 to 50 microliters, or preferably 1 to 20 microliters, or more preferable 1 to 10 microliters.

The following examples are provided for purposes of illustration of embodiments only and are not intended to be limiting. The reagents, chemicals and other materials are presented as exemplary components or reagents, and various modifications may be made in view of the foregoing discussion within the scope of this disclosure. Unless otherwise specified in this disclosure, components, reagents, protocol, and other methods used in the system and the assays, as described in the Examples, are for the purpose of illustration only.

### Example 1

### Materials and Instruments

Assay Cartridge and Instrument.

The system described in the examples here combined single-use disposable assay cartridges with a reader instrument. Fluorescence immunoassays were illuminated and captured and imaged using a multi-mode planar waveguide technology. Various types of planar waveguides have been used in biosensor and immunoassay applications for decades, and are the subject of several technical reviews. Briefly, a light source (e.g., a laser) was directed into a waveguide substrate. The present system uses a planar waveguide system as disclosed, for example, in U.S. Patent Application Publication No. 2010/0220318 entitled “Waveguide with Integrated Lens” as filed 12 Nov. 2009, and U.S. Patent Application Publication No. 2011/0049388 entitled “Planar Optical Waveguide with Core of Low-Index-of-Refraction Interrogation Medium” as filed 9 Nov. 2010, which applications are incorporated herein by reference in its entirety.

The cartridge used in the Examples is a simple single channel assembly based on an injection-molded, plastic planar waveguide with an integrated lens to facilitate light insertion therein. A double-sided adhesive gasket is used to define a fluidic channel for containment of the processed sample. The gasket also binds the planar waveguide to an injection molded upper component. The upper component provides fluid input and output ports, as discussed in U.S. Patent Application Publication No. 2012/0071342 filed Sep. 15, 2011, which is incorporated herein in its entirety. An absorbent pad above the output port on the upper component may be enclosed with a snap-in plastic lid, making cartridge fluids self-contained, thereby minimizing biohazard. Laser welding may further provide a lower cost, potentially more rapid alternative to gasket adhesion.

Samples may be processed before and after loading onto the cartridges on benchtop at ambient temperature, which in this study was approximately 20 to 25° C. Since the assay procedure may be performed independently of the reader instrument, sample cartridges can be batch processed in parallel.

### Example 2

### Competitive Assay

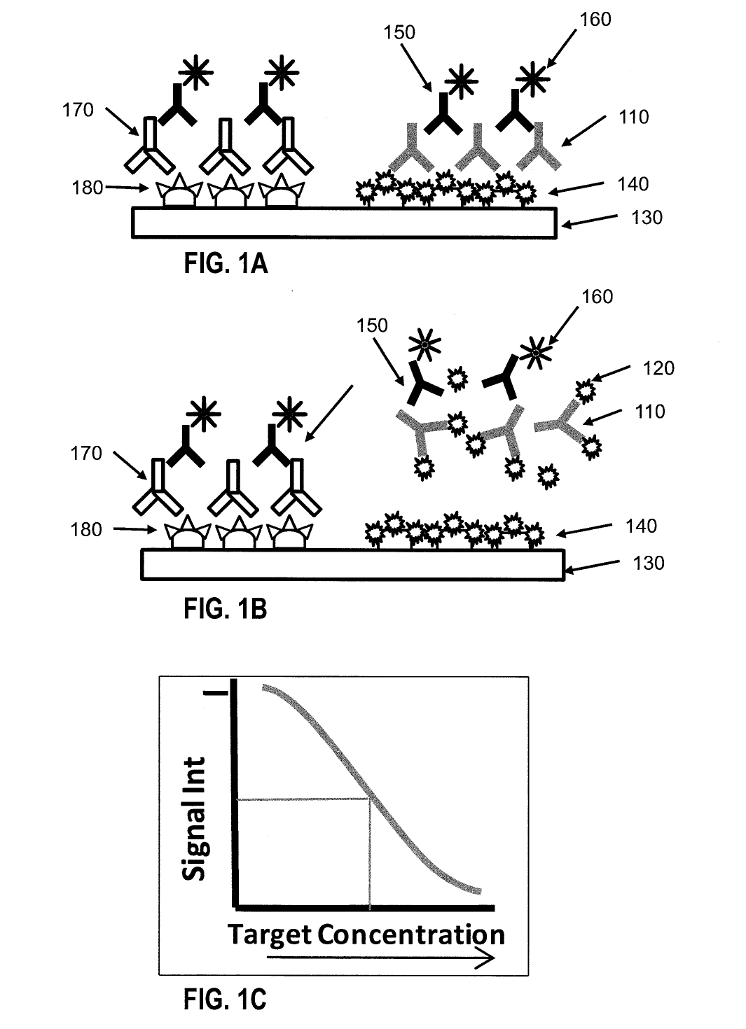

FIG. 1 shows a diagrammatic representation of an indirect competitive assay technique. According to FIG. 1, a primary anti-B antibody 110 may be mixed with a sample containing a target analyte B 120. A device having a surface 130 serves as the platform for the assay. Capture molecules 140 are immobilized on the surface 130. By way of example, FIG. 1 shows antigen B (same as target analyte B) as the capture molecule. A secondary antibody 150 with excitable tag 160 recognizes the primary antibody 110. When exciting light is shed on the spot on the surface, the excitable tag emits light signal which has intensity that is proportional to the amount of excitable tags attached to the spot. When no target analyte B is present in the sample, all of the anti-B antibodies 110 bind to the capture molecule 140 (FIG. 1A). When target analyte B 120 is present in the sample, target analyte B 120 competes against capture molecule 140 in binding with the labeling molecules 110 thereby reducing the amount of labeling molecules 110 that are attached to the capture molecule 140 (FIG. 1B). Thus, the signal intensity obtained from the spot is inversely proportional to the amount of target analyte in the sample (FIG. 1C). By contrast, because no antigen A is present in the sample, the anti-A antibody 170 binds to the immobilized antigen A 180 without any competition from the free antigen A in the sample (FIG. 1B).

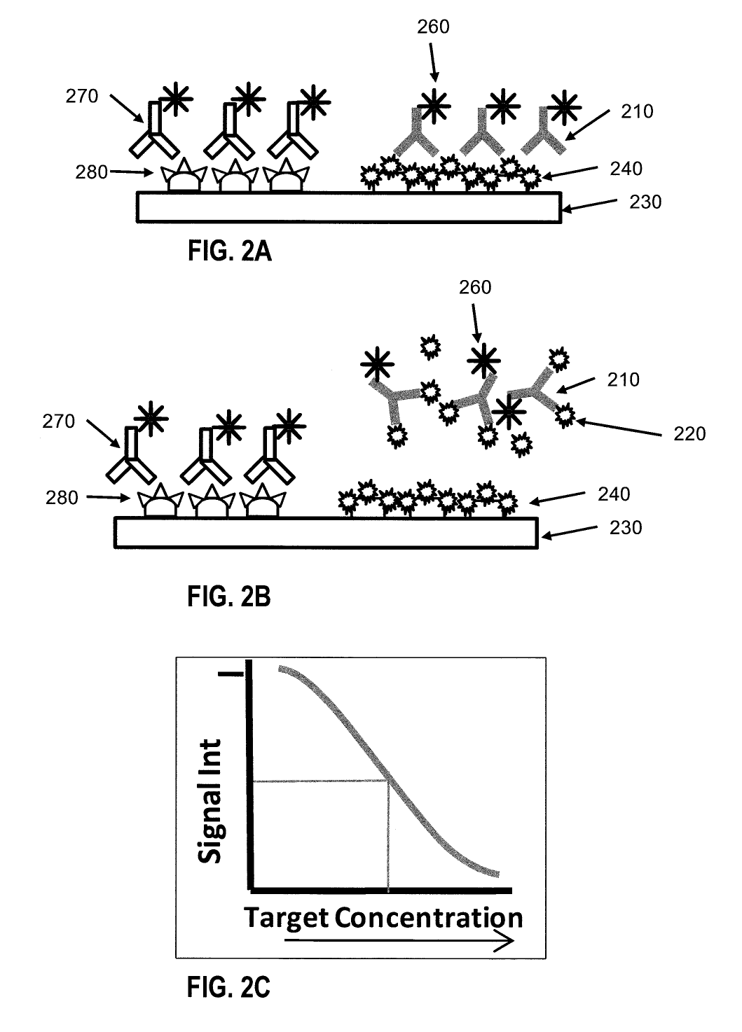

FIG. 2 shows a diagrammatic representation of a direct competitive assay technique, in accordance with an embodiment of the present disclosure. According to FIG. 2, a primary anti-B antibody is used as the labeling molecule 210, which may be mixed with a sample containing a target analyte B 220. A device having a surface 230 serves as the platform for the assay. Capture molecules 240 are immobilized on the surface 230. In one embodiment, surface 230 may be a waveguide. In another embodiment, surface 230 may be a planar waveguide having a refractive volume which optically couples light to the planar waveguide. By way of example, FIG. 2 shows antigen B (same as target analyte B) as the capture molecule 240. The labeling molecule 210 (anti-B antibody) is pre-conjugated with an excitable tag 260. When exciting light is shed on a spot on the surface 230, the excitable tag 260 emits light signal having intensity that is proportional to the amount of excitable tags attached to the spot. When no target analyte B is present in the sample, all of the anti-B antibodies 210 bind to the capture molecule 240 (FIG. 2A). When target analyte B 220 is present in the sample, target analyte B 220 competes against capture molecule 240 in binding with the labeling molecules 210, thereby reducing the amount of labeling molecules 210 that are attached to the capture molecule 240 (FIG. 2B). Thus, the signal intensity obtained from the spot may be inversely proportional to the amount of target analyte in the sample (FIG. 2C). By contrast, because no antigen A is present in the sample, the tagged anti-A antibody 270 binds to the immobilized antigen A 280 without any competition from the free antigen A in the sample (FIG. 2B).

In one aspect, a dye-conjugated antibody may be used as the labeling molecule 210 in a competitive assay to detect a target analyte 220 in a sample. The sample may be a fluidic, aerosol, or solid sample. In another aspect, the sample may be a biological sample or a non-biological sample. In another aspect, the sample may be obtained from a human, from an animal, from a plant, or otherwise obtained from the environment, from a natural source, or from an industrial process. A solid sample may be converted into a fluidic sample by dissolving or suspending the solid sample in a liquid carrier (e.g., a solvent) that does not interfere with the assay. In one embodiment, the sample may be a blood sample, a urine sample or a saliva sample.

The target may be any molecule that is present in the sample. For example, the target (also referred to as “target molecule” or “target analyte”) may be a peptide, a polypeptide, a protein, an antibody, an antigen, a polysaccharide, a sugar molecule, an oligonucleotide, a polynucleotide, an inorganic molecule, an organic molecule, a cell, or combination thereof. In one aspect, each target may have a binding partner, which is an unlabeled labeling molecule or pre-labeled labeling molecule with an excitable tag. For purpose of illustration only, the labeling molecule 210 is shown as an antibody in FIG. 2. However, the labeling molecule may be an aptamer, a peptide, a polypeptide, a protein, an antibody, an antigen, a polysaccharide, a sugar molecule, an oligonucleotide, a polynucleotide, a synthetic molecule, or other molecular recognition element. In one embodiment, the labeling molecule may be pre-conjugated with a detectable tag. In another embodiment, the labeling molecule may be unlabeled and may be labeled with a detectable tag after binding with the target.

An assay device (such as a cartridge) having a surface may be used to receive the sample. In one embodiment, the labeling molecule 210 may be mixed with the sample to create a pre-mix (or mixture). The pre-mix may be applied to the assay device by spotting on the surface 230 of the device. A capture molecule 240 similar or identical to the target molecule 220 may be immobilized on the surface. This immobilized molecule may bind to the labeling molecule when the pre-mix is applied to the surface. The number of labeling molecules bound to the immobilized molecules on the surface may be calculated by using the strength of signals detected from the different spots on the surface. In one aspect, all binding sites on the labeling molecules that bind to the immobilized molecules are also capable of binding the target molecules. In another aspect, the immobilized molecules on the surface of the device and the target molecules in the sample may compete for binding with the labeling molecules. Thus, the number of labeling molecules bound to the immobilized molecules may be a function of the number of target molecules in the sample. In other words, the strength of the signal detected by the device may be inversely proportional to the total number of target molecules in the sample.

A standard curve may be created by using known amount of a molecule (referred to as a “calibrating molecule”) that is similar or identical to the target molecule. In one aspect, a series of calibrating samples containing varying amounts of the calibrating molecule may be prepared. The calibrating samples may be prepared to mimic various chemical, biological and/or physical properties of the actual test sample except for the absence of the target molecules. Each calibrating sample may be mixed with the labeling molecules to create a pre-mix, which is applied to the assay surface. The signals obtained from each calibrating sample may then be plotted against the concentration of the calibrating molecule to generate a standard curve.

To quantitate the amount of a target molecule in a test sample, the test sample may be pre-mixed with a labeling molecule to create a pre-mix. The pre-mix may then be applied to the assay device. The signal strength detected from the test sample may be compared against the standard curve to obtain the concentration of the target molecules in the test sample. It is to be recognized that certain systematic adjustment may be needed to obtain the actual concentration of the target molecules.

In one embodiment of the present disclosure, the target is an antigen (also referred to as “target antigen”), and the labeling molecule is an antibody, which is capable of binding to the target antigen. The antibody may be labeled with a tag, which may be, by way of example, a fluorescent dye, lanthanide, nanoparticle, microparticle, light-scattering particle, or other labeling molecules. In one aspect, a small molecule having the same property as the target antigen is immobilized on an assay surface. For example, the immobilized antigen may be a peptide with the same antigen epitope as a target antigen that will be detected in a sample. In another aspect, the immobilized antigen may be the same as the target antigen, or may be a fragment of the target antigen. In another aspect, the target antigen may be a fragment of the immobilized antigen.

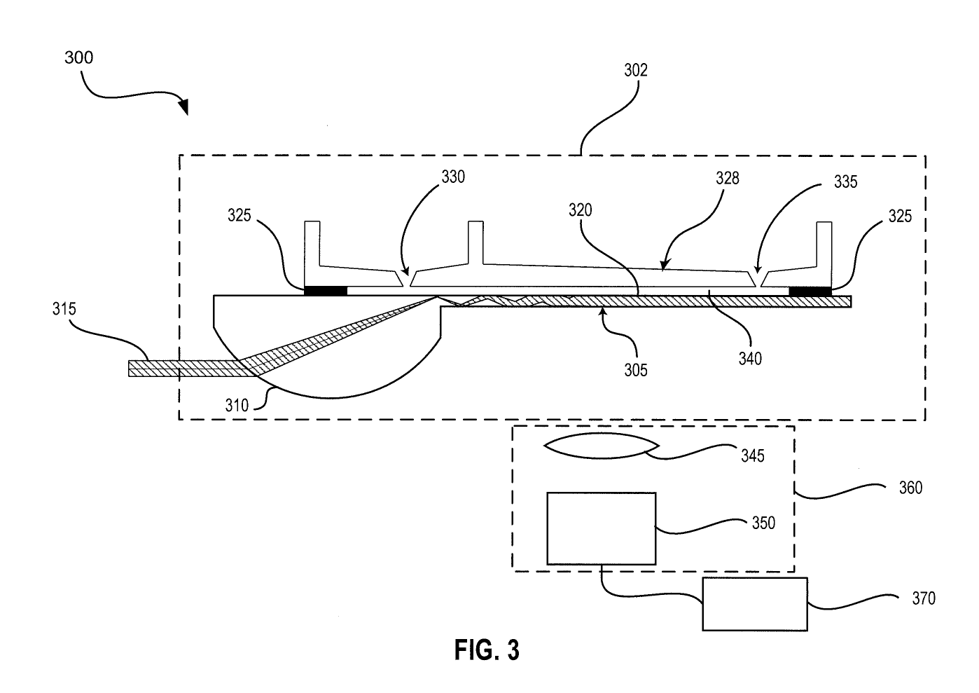

In another embodiment, an antibody specific to target antigen may be covalently labeled with a tag, such as a fluorescent dye. During the assay, the dye-labeled antibodies may be first mixed with a sample to create a pre-mix and the pre-mix may then be incubated on the assay surface with the immobilized antigen. If no target antigen is present in the sample, dye-labeled antibodies bind to the surface-bound antigens, and a significant amount of fluorescence signal would be observed in a detection system. If a significant amount of target antigen is present in the sample, dye-labeled antibodies bind to the target antigen in the sample, which inhibits, or “competes” with binding between the immobilized antigens and the dye-labeled antibodies. As a result, decreased amount of fluorescence signal would be observed. The fluorescent signal from the label may be detected via fluorescence imaging, using an evanescence illumination configuration such as shown in FIG. 3, and also as described in U.S. Patent Application Publication No. 2010/0220318.

In an equilibrium system such as antigen-antibody binding, the observed fluorescence signal may be inversely proportional to the amount of target antigen in the sample. Standard curves can be established that allow quantitative determination of target antigen concentration in a test sample. A specific example of a competitive assay is described in details below.

FIG. 3 shows an exemplary system for performing a rapid, simple assay for detecting one or more target molecules in a single biological sample, in accordance with an embodiment. FIG. 3 illustrates a cross-sectional view of an assay system 300, including a cartridge 302. Cartridge 302 includes a planar waveguide 305 with an integrated lens 310 suitable for use with the labeled antigen assay of FIG. 2, in accordance with the embodiment (See U.S. Patent Application Publication No. 2010/0220318). An illumination beam 315 is inserted into planar waveguide 305 through integrated lens 310. Illumination beam 315 may be provided, for example, by a laser with an appropriate wavelength to excite fluorescent labels at an assay surface 320. Other appropriate forms of illumination, either collimated or uncollimated, may also be used with assay system 300. Integrated lens 310 is configured to cooperate with planar waveguide 305 such that illumination beam 315, so inserted, is guided through planar waveguide 305 and may illuminate assay surface 320 by evanescent light coupling. Assay surface 320, an upper component 328, which includes an inlet port 330 and an output port 335, cooperate to define a fluidic sample chamber 340. Assay surface 320 and upper element 328 can be bonded via a channel-defining adhesive gasket 325 or via direct bonding methods such as laser welding, ultrasonic welding, or solvent bonding.

Appropriate molecules (e.g., immobilized antigen described above), may be bound to assay surface 320 such that when a biological sample and labeled detect reagent (also known as “labeling molecule”, e.g., labeled antibodies) are added to the fluidic sample chamber 340, a target analyte (or “target molecule”), if present in the sample, binds with the detect reagent in solution and prevents the detect reagent from binding with the immobilized molecules on the assay surface 320. If the target analyte is not present, the detect reagent binds to immobilized molecules on the assay surface 320. In this configuration, the fluorescence signal is inversely proportional to the concentration of target analyte in the sample. For instance, in the absence of the target analyte, fluorescence signal is highest. By contrast, a high concentration of the target analyte in the samples may result in low or no fluorescence.

As an example, collection and filtering optics 345 may be used to capture the fluorescence signal from assay surface 320. A signal corresponding to the fluorescence so captured may then be directed to an imaging device 350, such as a CCD or CMOS camera. More particularly, methods to measure signal intensities from the spots may include but are not limited to CCD or CMOS camera image acquisition from laser excitation through a planar waveguide, CCD or CMOS camera image acquisition from appropriately filtered white light excitation, camera image acquisition from chemiluminescence light generation, laser scanning with photo multiplier tube (PMT) collection for pseudo image generation, or other methods to record specific signal associated with spots on the array. In one aspect, the intensity of the signal may be measured by the light signal obtained directly from the spot. In another aspect, the intensity of the signal may be measured by taking an image of the array of spots and scanning the image to compare the intensity of each spot on the image.

In a further embodiment, the disclosed system and method may be used for rapid, simple detection of multiple target analytes in a single sample. Two or more different molecules may be immobilized to the assay surface, such as in stripes or spots in an array format using printing technology, thereby creating a spatially-localized set of parallel assay locations. The corresponding fluorescent dye-labeled detect reagents may be mixed into a single cocktail, referred to herein as a “pre-mix” or a “labeled detect reagent mix.” The combination of a sample, labeled detect reagent mix, and immobilized molecules on the assay surface 320 may lead to the formation of multiple physically separated complexes on the assay surface. Illumination of assay surface 320 results in spatially-localized fluorescence signal that may be read with a detection system 360 including collection and filtering optics 345, imaging device 350, and computer 370. Computer 370 may be integrated into the detection system instrument (e.g., single board computer). Alternatively, the computer 370 could be an external device.

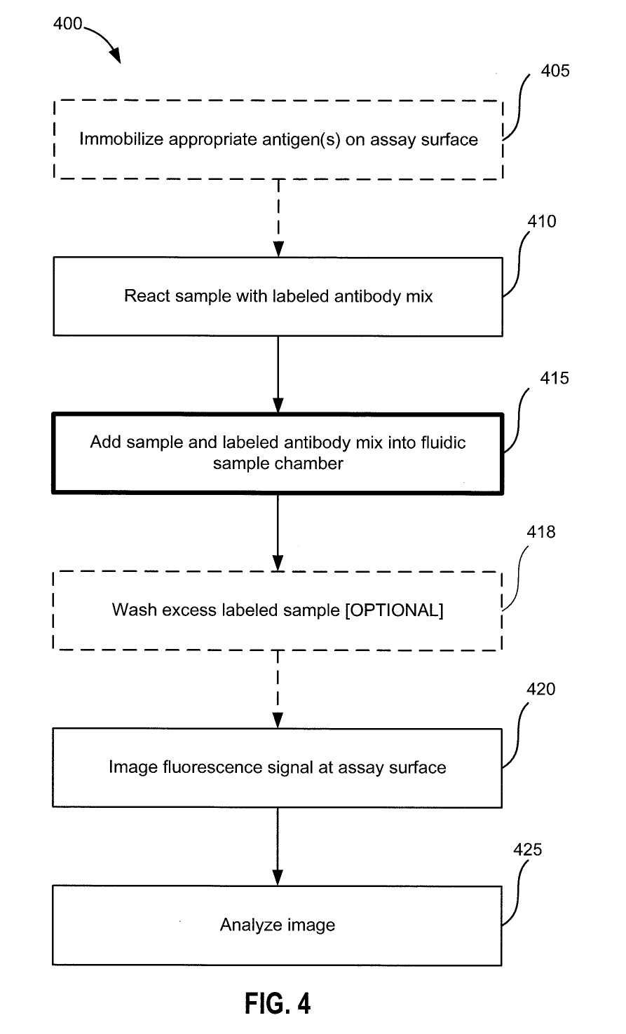

FIG. 4 shows a flow chart, summarizing an exemplary competitive assay process flow, in accordance with an embodiment. An assay process 400 may begin with an antigen immobilization step 405, in which one or more appropriate antigens as well as potentially positive and negative controls are immobilized on an assay surface, such as assay surface 320 of FIG. 3. Step 405 may be performed, for example, by the manufacturer of the assay system rather than the assay system user.

Assay process 400 then proceeds to a step 410, in which a sample, and a labeled detect reagent mix is added to a fluidic sample chamber, such as fluidic sample chamber 340. The labeled antibody mix may be provided by the assay system manufacturer or custom-formulated by the assay system user. In step 415 the pre-mix of sample and labeled antibody created in step 410 may be added to the sample chamber 340 of FIG. 3. Optionally, excess detect reagent mix may be washed away from assay surface 320 in an optional step 418. The fluorescence signal at the assay surface is then imaged by the assay system in a step 420, and then the captured image may be analyzed in a step 425. The example below provides an exemplary demonstration of assay process 400.

The assay described herein may be further simplified. In an embodiment, the labeled detect reagent mix may be immobilized within fluidic sample chamber 340 using conventional methods such as lyophilization. For example, the labeled detect reagent mix may be lyophilized along with sugar-based stabilizers at or near inlet port 330 of assay system 300. Upon sample introduction, the labeled detect reagent mix is rehydrated to be available to mix with any free target analyte in the sample. In another embodiment, unlabeled detect reagent may be lyophilized at or near inlet port 330 of assay system 300, and a labeled secondary detect reagent may be lyophilized and spotted on the same spots as the capture molecules, or the labeled second detect reagent may be lyophilized and spotted on the path between the sample inlet and the capture molecule spot. After the detect reagent(s) binds to the one or more target analytes in the sample, as the mixture moves down the path, it makes contact with the labeled secondary detect reagent before making contact with the capture molecule(s). The secondary detect reagent may be a secondary antibody conjugated with an excitable tag.

A further advantage of this embodiment is that the sensitivity of assay system 300 may allow elimination of subsequent wash steps. In particular, when using planar waveguide illumination, the evanescent field is localized within a few hundred nanometers of the assay surface for visible light illumination. Consequently, fluorescent dye in the bulk solution of fluidic sample chamber 340 does not contribute to the fluorescence signal measured at detection system 360.

The result is a true single step assay: a sample is added to cartridge 302, which is then imaged on detection system 360 in step 420 and subsequently analyzed in step 425. Alternatively, a wash step 418 may potentially yield improved signal-to-background performance in the assay and may therefore be useful in certain assay applications. Several methods for the wash step may be envisioned. For example, this step may be a simple wash buffer addition introduced by the user from a dropper bottle. Alternatively, the final wash buffer may be stored on-board the device, such as in a blister pack that is either deployed by the user or automatically by activation in the detection system.

It is noted that the workflow outlined in FIG. 4 is only exemplary. Other embodiments may have different sequences of steps or additional modifications.

In another embodiment, fluidic sample chamber 340 in cartridge 302 may be specifically designed to improve assay performance by controlling fluid flow rates over the assay surface. Static incubations in small fluidic channels generally have limits of detection set by mass transport limitations (e.g., diffusion) in the system. By engineering the fluidic sample chamber geometry (i.e., length, width, height, shape) and surface energies, sample flow rate over the assay surface may be optimized for improved assay performance.

It is further noted that the above embodiments are described in terms of antibody-antigen immunoassays. The competitive assay concept described here, however, is not restricted only to antibody-antigen immunoassays. The competitive assay approach and detection system described herein may be used, for example, with nucleic acid (e.g., DNA, RNA) based assays and cell-based assays, and may be used to quantitate in a sample the amount of a peptide, a polypeptide, a protein, an antibody, an antigen, a polysaccharide, a sugar molecule, an oligonucleotide, a polynucleotide, an inorganic molecule, an organic molecule, a cell, or combination thereof.

Samples may be processed before and after loading onto the cartridges on benchtop at ambient temperature, which in this study was approximately 20 to 25° C. Since the assay procedure may be performed independently of the reader instrument, sample cartridges can be batch processed in parallel.

In an embodiment, a labeled antibody is used as the detect reagent in a competitive binding assay. Briefly, a molecule having the same antigenic epitope as the target analyte molecule was covalently bound to an assay surface. An antibody, specific to the surface bound antigen and the target analyte molecule, is covalently labeled with fluorescent dye and mixed with a sample to form a pre-mix. The pre-mix is added to the assay surface where it is incubated with the surface bound antigens. In the absence of target analyte, labeled antibody incubated on an assay surface the labeled antibody detect reagent would bind to surface bound antigen, where it could be detected via fluorescence imaging as described above. If a sample contained target analyte, some or all of the target analyte molecules would bind the labeled antibody detect reagent which would be unavailable to bind the surface antigen when incubated on the assay surface. Thus, lower recorded signal as compared to a control sample containing no free antigen indicates the presence of the target analyte (or “target antigen”) in a sample. Further, the relative drop of signal is proportionate to the concentration of antigen in the sample, and can be used as a quantitative method of small molecule detection.

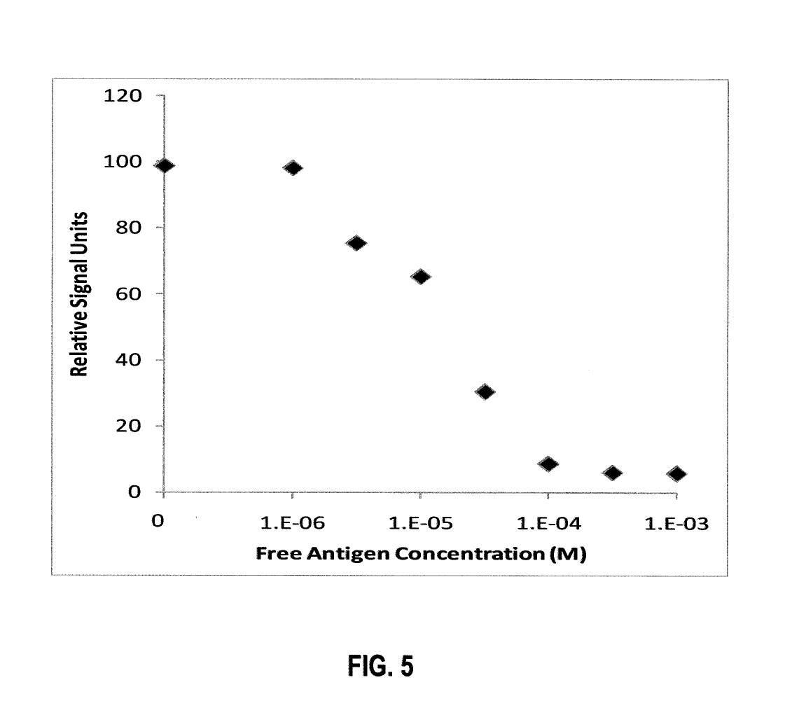

As a specific example, Saxotoxin-OVA conjugate is printed to an assay surface. Polyclonal Rabbit anti-Saxotoxin antibody had been labeled with Alexa647 fluorescent dye. A sample containing labeled anti-Saxotoxin antibody is incubated on the assay device, followed by fluorescent signal acquisition yielding relative signal of 100. No rinsing steps are performed, emphasizing the single step utility of the assay. Samples containing serially diluted concentrations of Saxotoxin-OVA are subsequently incubated on assay devices and fluorescence signal acquired, with recorded signal, relative to the no Saxotoxin-OVA sample, proportionate to concentration of Saxotoxin-OVA in the samples (see FIG. 5). As shown in FIG. 5, when the concentrations of Saxotoxin-OVA are between 1×10−6 M and 1×10−4 M, the signal strength is inversely proportional to the concentration of the Saxotoxin-OVA (or Saxotoxin) in the sample. The result demonstrates the feasibility of a simple one-step fluorescence competitive small molecule detection assay.

Changes may be made in the above methods and systems without departing from the scope hereof. It should thus be noted that the matter contained in the above description or shown in the accompanying drawings should be interpreted as illustrative and not in a limiting sense. The following claims are intended to cover generic and specific features described herein, as well as statements of the scope of the present method and system, which, as a matter of language, might be said to fall therebetween.

Although each of the aforedescribed embodiments have been illustrated with various components having particular respective orientations, it should be understood that the system as described in the present disclosure may take on a variety of specific configurations with the various components being located in a variety of positions and mutual orientations and still remain within the spirit and scope of the present disclosure. For example, it should be noted that the present configuration may be applicable for systems in which the core refractive index is greater than the refractive indices of the substrates, such as if a solid core material is used, as long as the surrounding medium refractive index is less than those of the substrates. Additionally, in the various figures described above, the gasket may be eliminated and replaced with direct laser welding of first and second substrates. Furthermore, suitable equivalents may be used in place of or in addition to the various components, the function and use of such substitute or additional components being held to be familiar to those skilled in the art and are therefore regarded as falling within the scope of the present disclosure. Therefore, the present examples are to be considered as illustrative and not restrictive, and the present disclosure is not to be limited to the details given herein but may be modified within the scope of the appended claims.

What is claimed is:

1. A method for analyzing one or more target analytes in a sample, the method comprising:
(a) forming a mixture by contacting said sample with one or more types of labeling molecules, wherein at least one of said labeling molecules binds to at least one of said target analytes if said sample contains said one or more target analytes;
(b) contacting the mixture of step (a) with one or more types of capture molecules immobilized on a surface of a device, wherein said one or more types of capture molecules bind to said one or more types of labeling molecules; and
(c) measuring intensity of a first signal emitted from the surface of said device.

2. The method of claim 1, wherein each of said labeling molecules comprises an excitable tag.

3. The method of claim 1, wherein the presence of a specific target analyte in a sample inhibits binding of at least one labeling molecule to at least one capture molecule, such that the presence of said target analyte results in a decreased first signal intensity as measured in (c) relative to intensity obtained from a sample containing no target analyte.

4. The method of claim 3, wherein said decrease in first signal intensity is quantitatively related to the amount of said specific target analyte in the sample.

5. The method of claim 1, wherein said labeling molecule is an affinity binding molecule selected from the group consisting of an antibody, an aptamer, a protein, a small organic molecule, a carbohydrate, a lipid molecule, a polynucleotide, and combination thereof.

6. The method of claim 5, wherein at least one of said labeling molecules is an antibody.

7. The method of claim 1, wherein said mixture comprises two or more different types of labeling molecules.

8. The method of claim 1, wherein said capture molecules are immobilized in a spatial array of spots on said surface, said spatial array comprising at least two spots, wherein each spot contains capture molecules of one specific type.

9. The method of claim 8, wherein said measuring step (c) comprises measuring said first signal from each spot of said spatial array, wherein signal intensities from each spot are measured independently.

10. The method of claim 8, wherein said measuring step (c) comprises obtaining an image of said spatial array.

11. The method of claim 1, further comprising a step (d) of comparing said intensity of said first signal against intensity of a second signal obtained from a calibrating molecule having known amount.

12. The method of claim 11, wherein the intensity of said first signal is compared against the intensity of a plurality of second signals obtained from calibrating molecules having an increasing or decreasing series of known amounts.

13. The method of claim 12, wherein the intensity of said second signals obtained from said calibrating molecules is used to construct a standard curve.

14. The method of claim 11, wherein said calibrating molecules and said target analytes have substantially identical binding affinity with said labeling molecules.

15. The method of claim 11, wherein said calibrating molecules are identical to said target analytes.

16. The method of claim 1, wherein said capture molecules and said target analytes have substantially identical binding affinity with said labeling molecules.

17. The method of claim 1, wherein said capture molecules and said target analytes bind to the same binding site on said labeling molecules.

18. The method of claim 1, wherein said capture molecules are a member selected from the group consisting of a protein, a small organic molecule, a carbohydrate, a lipid molecule, a polynucleotide and combination thereof.

19. The method of claim 1, wherein at least one of said capture molecules is an antigen.

20. The method of claim 1, wherein said device comprises a first substrate, a second substrate, and a refractive volume,
said first substrate comprising a planar waveguide, wherein said planar waveguide has a first outer surface and a first inner surface,
said second substrate comprising a second outer surface and a second inner surface, wherein said first substrate and said second substrate are positioned such that at least a section of said first inner surface and a section of said second inner surface are apart from each other at a distance wherein said section of said first inner surface and said section of said second inner surface at least partly define a sample chamber for confining at least a portion of said sample, and
wherein said refractive volume optically couples light to said planar waveguide.

21. The method of claim 20, wherein said capture molecules are attached to said first inner surface of said planar waveguide.

22. The method of claim 21, wherein said device is a cartridge, and wherein said measuring step (c) comprises positioning said cartridge in a reader instrument, wherein said instrument directs a light beam from a light source into said refractive volume such that the light beam is focused on the first inner surface of the planar waveguide at a non-zero, internal propagation angle relative to the first inner surface for all light within the light beam, wherein said light beam excites an excitable tag bound to said labeling molecule, said labeling molecule being bound to said capture molecules on said first inner surface of said planar waveguide.

23. The method of claim 22, wherein said measuring step (c) further comprises capturing the light signal emitted by said excitable tag and analyzing the light signal.

24. The method of claim 1, wherein sample is in a liquid form and the dynamic range of said method is in the range of about 0.001 ng target analyte per ml to about 1,000,000 ng target analyte per ml.

25. The method of claim 1, wherein said labeling molecules comprise at least two different antibodies with substantial but different affinities to the same target analyte.

26. The method of claim 11, wherein the target analytes and the calibrating molecules are different, and capture molecules for both antibodies against the target analytes and antibodies against the calibrating molecules are immobilized at the same spot on said surface.

27. The method of claim 1, wherein said device is a cartridge having a sample inlet, and said labeling molecules are incorporated in the cartridge, wherein a sample containing unknown concentration of target analytes contacts said labeling molecules prior to contacting with said immobilized capture molecules.

28. The method of claim 27, wherein said labeling molecules are incorporated in the cartridge in a stable, dry formulation, wherein the addition of sample rehydrates said labeling molecules.

29. The method of claim 1, wherein said step (a) and step (b) occur in the same device.

30. The method of claim 1, wherein step (a) precedes step (b) on the device and no operator intervention is required between step (a) and step (b).

31. The method of claim 1, wherein steps (a)-(c) are performed without a wash step.

32. A method for analyzing one or more target analytes in a sample, the method comprising:
(a) adding said sample into a device,
(b) allowing said sample to contact one or more types of labeling molecules, wherein at least one of said labeling molecules binds to at least one of said target analytes if said sample contains said one or more target analytes;
(c) contacting the mixture of step (b) with one or more types of capture molecules immobilized on a surface of said device, wherein said one or more types of capture molecules bind to said one or more types of labeling molecules; and
(d) measuring intensity of a first signal from the surface of said device,
wherein said labeling molecules are incorporated in said device as a stable, dry formulation, and wherein said sample rehydrates said labeling molecules in step (b).

33. A method for analyzing one or more target analytes in a sample, the method comprising:
(a) forming a mixture by contacting said sample with one or more labeling molecules, said mixture comprising said one or more labeling molecules and said one or more target analytes, wherein at least one of said labeling molecules binds to at least one of said target analytes;
(b) contacting the mixture of step (a) with one or more capture molecules, said capture molecules being immobilized on a surface of a device, wherein at least one of said labeling molecules binds to at least one of said capture molecules when said mixture is in contact with said capture molecules; and
(c) measuring intensity of a first signal emitted by said labeling molecules that are bound to said capture molecules, wherein said labeling molecules comprise an excitable tag or are labeled with an excitable tag,
wherein said device comprises a first substrate, a second substrate, and a refractive volume,
said first substrate comprising a planar waveguide, wherein said planar waveguide has a first outer surface and a first inner surface,
said second substrate comprising a second outer surface and a second inner surface, wherein said first substrate and said second substrate are positioned such that at least a section of said first inner surface and a section of said second inner surface are apart from each other at a distance wherein said section of said first inner surface and said section of said second inner surface at least partly define a sample chamber for confining at least a portion of said sample, and
wherein said refractive volume optically couples light to said planar waveguide.
